(19) United States
(12) Patent Application Publication
(10) Pub. No.: US 2005/0043396 A1
(43) Pub. Date: Feb. 24, 2005

(54) 5-Benzoylamino-1,3-dioxacyclanes, the method for preparing the same and their use as PKC inhibitor

(76) Inventors: Hayley Zen, Beijing (CN); Shiqi Peng, Beijing (CN); Lanrong Bi, Beijing (CN); Ming Zhao, Beijing (CN); Chao Wang, Beijing (CN)

Correspondence Address: PALMER & DODGE, LLP;KATHLEEN M. WILLIAMS, 111 HUNTINGTON AVENUE, BOSTON, MA, 02199, US

(21) Appl. No.: 10/674,148

(22) Filed: Sep. 29, 2003

(30) Foreign Application Priority Data
Mar. 6, 2003 (CN) 03119229.7

(51) Int. Cl. A61K 31/335

(52) U.S. Cl. 514/452

(57) ABSTRACT

The present invention discloses a series of benzoylamino-1,3-dioxacyclane compounds, of which compounds 1-21 were prepared via transacetalisation reaction between N-benzoylaminoglycol and 1,1,3,3-tetramethoxypropane; while compounds 22-48 were prepared via stereospecific acetalisation reaction between N-benzoylamino glycol and aromatic aldehyde, and if necessary, the nitro groups were reduced and further be salified with propane diacid and L-Arg or L-Lys. These compounds possess the structural type of PKC inhibitor and positive anti-inflammatory effect, and can be applied in medical fields as PKC inhibitor for corresponding therapy.

### FIELD OF THE INVENTION

The present invention relates to cycloacetals (5-benzoylamino-1,3-dioxacyclanes) prepared via transacetalization of N-benzoylaminoglycol and 1,1,3,3-tetramethoxypropane, the use as PKC inhibitor, and more particularly, the present invention relates to transacetalization of N-benzoylaminoglycol and 1,1,3,3-tetramethoxypropane and the cycloacetal products (i.e. 5-benzoylamino-1,3-dioxacyclane compounds), and the stereospecific transacetalization of N-benzoylaminoglycol and 1,1,3,3-tetramethoxypropane, and acetalization of N-benzoylaminoglycol and aromatic aldehyde, and the stereospecific products (i.e. 5-benzoylamino-1,3-dioxacyclane compounds), and furthermore, the use of these products as PKC inhibitor.

### BACKGROUND OF THE INVENTION

Protein kinase C (PKC) derives from the kinase family, and can be found in almost each tissue. The activity of PKC relates to many kinds of important physiological processes, such as contraction of muscle, release of neurotransmitter, activating platelet, and function of growth factor and hormone, and pathologic process, such as cancer, inflammation, myocardial ischemia and multidrug resistance related to ischemia/reperfusion injury (Basu A, The potential of protein kinase C as a target of anticancer treatment, Pharmac. Ther. 1993, 59, 257-280; Weinstein IB, Begemann M.0. Disorders in cell circuitry associated with multistage carcinogeneses: exploctable targets for cancer prevention and therapy, Clin Cancer Res, 1997, 3, 2696-2702). The structural characters of exogenous PKC agonists, phorbol ester, teleocidin, ingenol, and endogenous PKC agonist diacetylglycerine were compared carefully (Wender P. A., The chemistry-medicine continuum: Synthetic, computer, spectroscopic and biological studies on new chemotherapeutic leads, Pure. & Apll. Chem, 1998, 70 (3), 539-546), and the structural characters of competitive compound template based on pentacylic lactone were also studied in detail. (Lee J, Conformationally constrained analogues of diacylglycerol, 10. Ultrapotent protein kinase C ligands based on a chiral 5-disubstituted tetrahydro-2-furanone template, J. Med. Chem, 1996, 39, 29-35; Lee J, Conformationally constrained analogues of diacylglycerol, 12. Ultrapotent protein kinase C ligands based on a chiral 5-disubstituted tetrahydro-2-furanone template, J. Med. Chem., 1996, 39, 36-45), from which the potential of cycloacetals as lead structure of PKC inhibitors was recognized.

### SUMMARY OF THE INVENTION

A common method to synthesis cycloacetal compounds was published in 1989, as well as the special stability of these compounds (Peng S Q, Winterfeldt E, Synthesis of malonaldehyde monoacetals, Liebigs Ann. Chem., 1989, 1045-1047). The transacetalization of N-benzoylaminoglycol and 1,1,3,3-tetramethoxypropane was published in 2000, as well as the cycloacetal compounds (Lan rong Bi, Ming Zhao, Chao Wang, Shiqi Peng, Stereoselective transacetalization of 1,1,3,3-tetramethoxypropane and N-benzoylaminodiols, Eur. J. Org. Chem, 2000, 2669-2676).

Based on the knowledge of cycloacetals as structural types of the PKC inhibitors, the PKC inhibitor model recognized (i.e. model of the ear swilling of mice) was adopted to evaluate the activity of cycloacetals 1-21 as PKC inhibitor. In the present invention, the apparent anti-inflammatory effects of cycloacetals were observed via oral administration of 1 time per day for 3 successional days under the dosage of 30 mg/kg, or under the dosage of 30 mg/kg or 10 mg/kg for 1 time, which indicated that PKC inhibitor structural types are positively formed by cycloacetals 1-21. In fact, as comparing anti-inflammatory effects of the cycloacetals, no significant advantage of anti-inflammatory effect was found when R is aldehyde group or cycloacetal; while no significant advantage of anti-inflammatory effect was found when the mother nuclide is heptacylic ring or octacyclic ring, and the water-solubility of compounds 1-21 was not satisfying.

According to the analysis of the structure-activity relationship, easier preparation and better activity can be achieved by substituting the substituent at the 2 position of cycloacetal compound with aromatic group. Based on the results, in the present invention, benzoylaminoglycol (including optically active benzoylaminoglycols) was prepared using L-amino acid as raw material via methyl esterification, benzoylation, and reduction reactions, and then via cyclization with p-nitrobenzaldehyde or phenylacrylaldehyde, in which the nitro groups in cyclized products were reduced to amino if necessary, and the reduced products react with propane diacid and basic amino acid successively to provide the water soluble complex salts if further necessary. 2-Phenyl-cycloacetal compounds and the corresponding complex salts 22-48 were then prepared, in which the basic amino acid can be L-Arg or L-Lys.

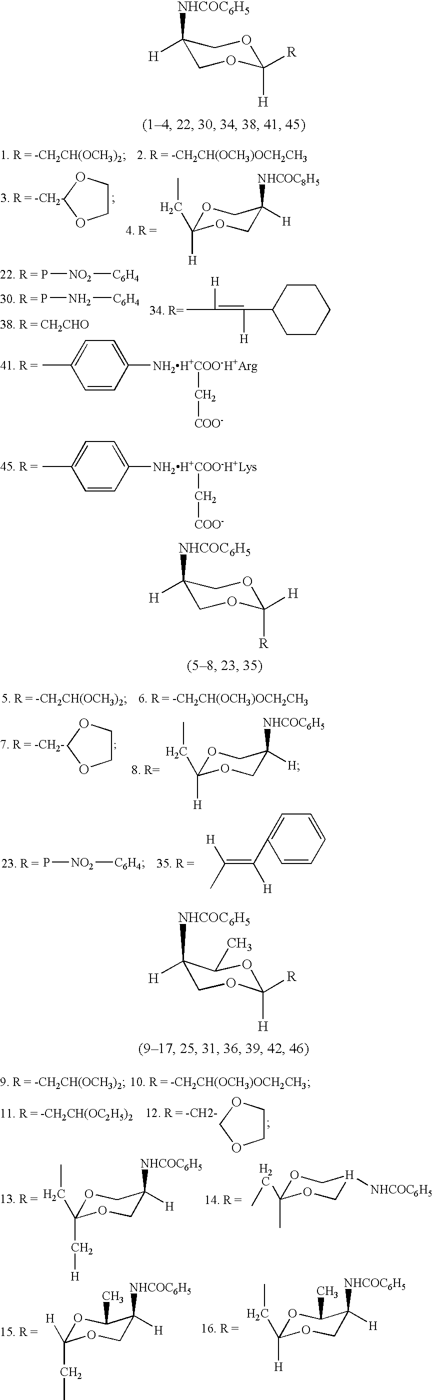

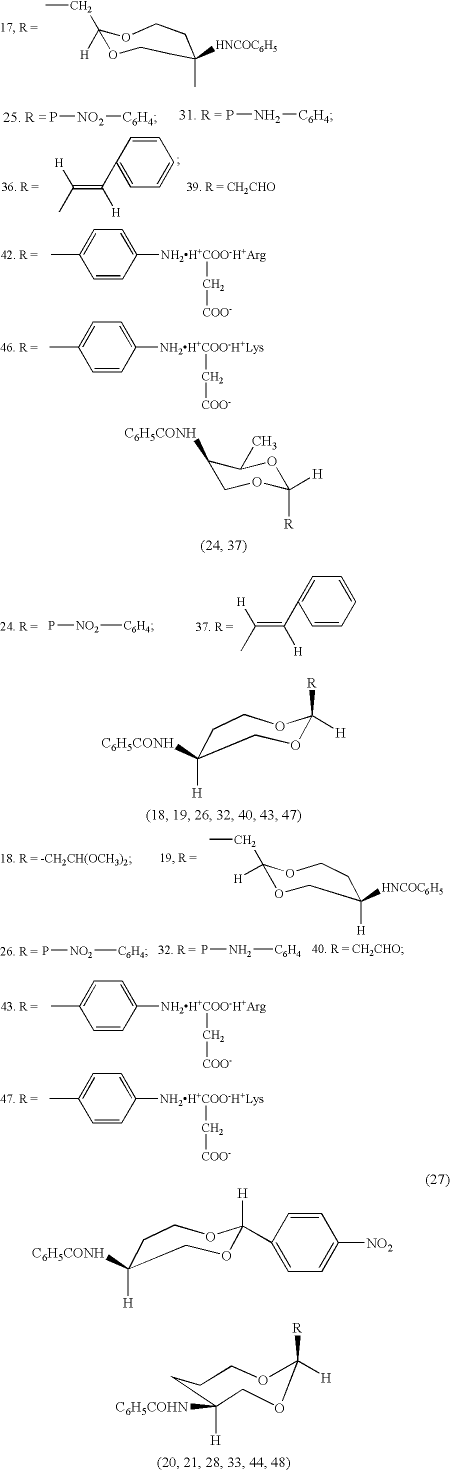

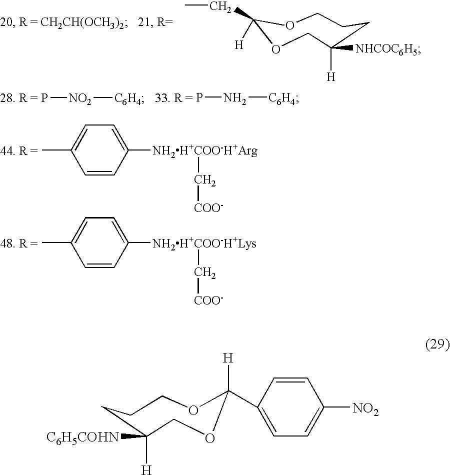

Following the synthetic route depicted below the L-amino acids were treated with methanol and sulfoxide chloride to convert into the corresponding L-amino acid methyl esters II (98% yield) in the present invention. The methyl esters IIa-d were acylated by use of benzoyl chloride and N-benzoyl-L-amino acid methyl esters of IIIa-d were obtained (81% yield). Using sodium borohydride as the reduction agent the benzoylated methyl esters were reduced to N-benzoylaminoglycols IVa-d successfully (97% yield).

| | |
|---|---|
| 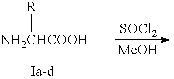 | 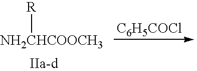 |

| | |
|---|---|
| 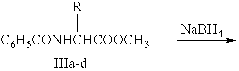 | 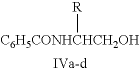 |

| | a | b | c | d |
|---|---|---|---|---|
| I | CH₂OH | CH(CH₃)OH | CH₂COOH | CH₂CH₂COOH |
| II [symbol] III | CH₂OH | CH(CH₃)OH | CH₂COOCH₃ | CH₂CH₂COOCH₃ |
| IV | CH₂OH | CH(CH₃)OH | CH₂CH₂OH | CH₂CH₂CH₂OH |

Following the synthetic route depicted below in the presence of anhydrous sodium sulfate and toluenesulfonic acid the cyclization of p-nitrobenzaldehyde and benzoylaminoglycol the corresponding cis/trans isomer 22/23, 24/25, 26/27, and 28/29 were prepared. According to ¹H NMR and NOE experiments compounds 22, 25, 26 and 28 were assigned to the 2,5-cis-disubstituted products, which were the main products, while compounds 23, 24, 27 and 29 were assigned to the 2,5-trans-disubstituted products, which were the minor products. Based on their conformation the 2,5-cis-disubstituted products were thermodynamically stable products, while the 2,5-trans-disubstituted products were kinetic controlled product.

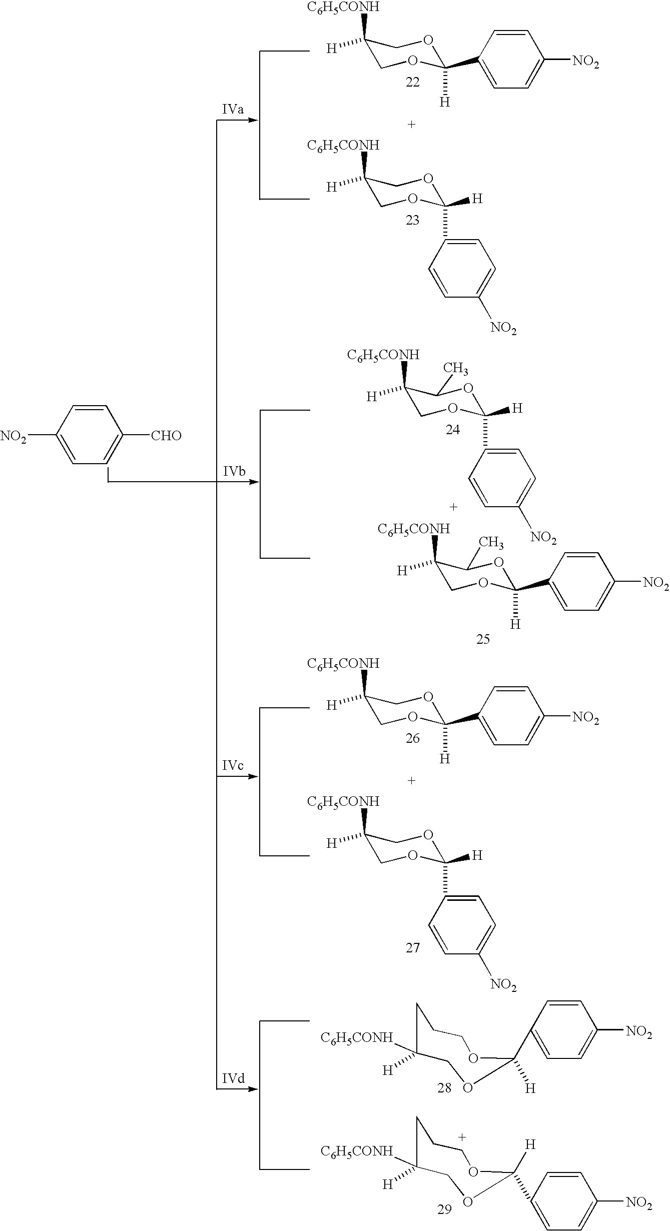

In this invention the synthetic route depicted below was suitable for the improvement of the water solubility and pharmacokinetics of the nitrified products, in which 2,5-cis-disubstituted products were reduced with sodium borohydride to produce the corresponding aminated products 30-33 which were treated with propane diacid and L-Arg or L-Lys in successively to produce the water soluble products 41-48.

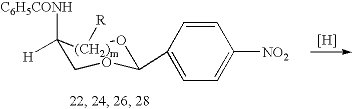

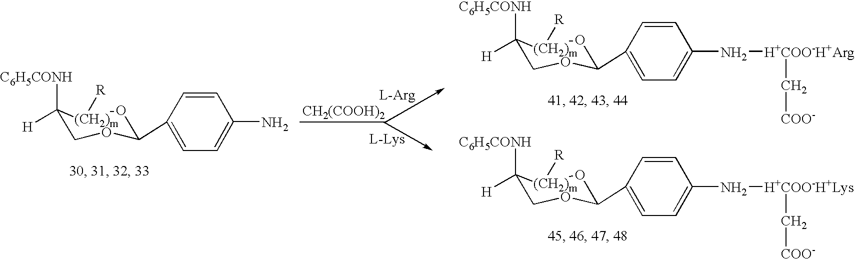

| | 22 | 24 | 26 | 28 | 30 | 31 | 32 | 33 | 41 | 42 | 43 | 44 | 45 | 46 | 47 | 48 |
|---|---|---|---|---|---|---|---|---|---|---|---|---|---|---|---|---|
| R | H | CH₃ | H | H | H | CH₃ | H | H | H | CH₃ | H | H | H | CH₃ | H | H |
| m | 0 | 0 | 1 | 2 | 0 | 0 | 1 | 2 | 0 | 0 | 1 | 2 | 0 | 0 | 1 | 2 |

Following the synthetic route depicted below in the presence of anhydrous sodium sulfate and toluenesulfonic acid the cyclization of trans-phenyl acrylaldehyde with 2-benzoylamino-1,3-propyleneglycol (IVa) or (2S,3R)-2-benzoylamino-1,3-propyleneglycol (IVb) provides the corresponding cis-trans isomers 34/35 and 36/37. According to ¹H NMR and NOE experiments compounds 34 and 36 were assigned to 2,5-cis-disubstituted products, which were the main products, while compounds 35 and 37 were assigned to 2,5-trans-disubstituted products, which were the minor products. Based on their conformation the 2,5-cis-disubstituted products were thermodynamically stable products, while the 2,5-trans-disubstitution products were kinetic controlled product.

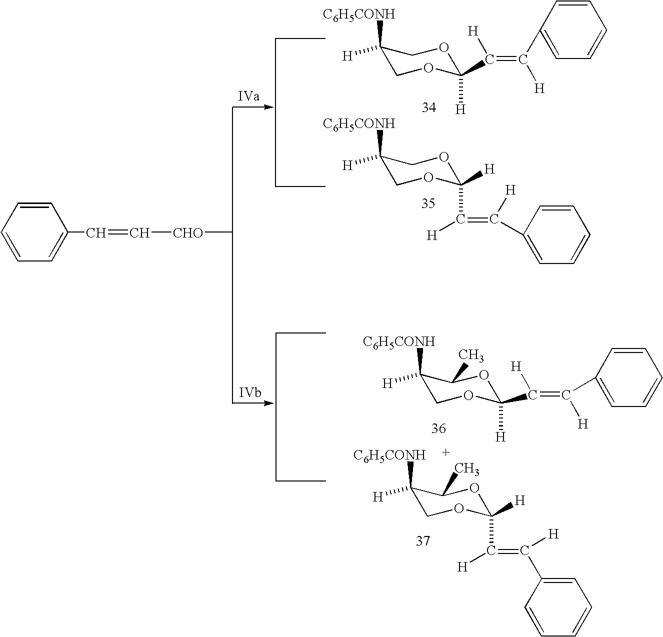

In the present invention the model of the ear swilling of mice was used to evaluate the anti-inflammatory activity of 5-benzoylamino-1,3-dioxscyclane compounds. The compounds of the present invention were suspended in CMC solution (5%) at the ratio of 0.3 mg/ml, while the positive control was the suspension of aspirin, and CMC solution (5%) at the ratio of 0.3 mg/ml, and the vehicle control was CMC solution (5%). Male Kunming species mice (from Animal Department of Beijing Medical University, grade 2) with weight about 20-25 g, which were randomly divided into treatment group, vehicle control group, and positive control group with 10 mice each group (food deprivation for 8 hours before testing, drinking unlimited) were administered orally once. 30 minutes after the administration, xylene (0.02 ml) was smeared on the left exterior auricular of the mice, and 4 hours later, the mice were killed by dislocation of cervical vertebra. Double circular pinnae were taken from two ears using 9 mm punching bear, and the swelling was determined by the weight variation of the two pinnae. It was shown that the compounds of the present invention possess excellent anti-inflammatory effect.

### DETAILED DESCRIPTION OF THE EMBODIMENTS

The present invention is described in detail with reference to the following examples. These examples, however, are intended to illustrate the present invention and should not be construed as limiting the scope of the present invention.

### EXAMPLE 1

### The Preparation of 2-p-nitrophenyl)-5-benzoylamino-1,3-dioxane

The suspension of 166 mg (0.85 mmol) of 2-benzoylamino-1,3-propyleneglycol (IVa), 128 mg (0.85 mmol) of p-nitrobenzaldehyde, 10 mg of p-nitrobenzenesulfonic acid and 100 mg of anhydrous sodium sulfate in 20 ml dichloromethane/tetrahydrofuran (5:1) was stirred at 50° C. for 12 hours, and the reaction mixture was cooled to room temperature when the TLC (chloroform/methanol, 25:1) indicated the disappearance of the raw material. The reaction mixture was neutralized using anhydrous Na₂CO₃ and then filtered, and the filtered cake was desiccated in vacuum desiccator after water washing. Thus 192 mg (69%) of cis-2-p-nitrophenyl-5-benzoylamino-1,3-dioxane (22) were obtained as colorless solid. The filtrate was evaporated under vacuum to offere 30 mg (11%) of trans-2-p-nitrophenyl-5-benzoylamino-1,3-dioxane (23) as colorless solid.

22: Mp, 220-224° C.; IR(KBr), ν/cm⁻¹=3270(NH), 3015(aromatic C═CH), 2920 and 2860 (CH and CH₂), 1620(C═O), 1605, 1580, 1545 and 1450 (aromatic C═C), 1380 (NO₂), 1190 and 1070(C—O—C), 800 (1,4-disubstituted 1656 and phenyl), ¹H NMR (DMSO): δ/ppm=4.205 (d, J=7.5H₂, 2H, NHCHCH₂O), 4.249 (d, J=7.5H₂,2H, NHCHCH₂O—), 4.310 (m, 1H, NHCHCH₂O—), 5.677 (S,1H, —O—CH—O—), 7.482 (t, J=6.6H₂, 2H, aromatic H), 7.557(t, J=6.6H₂, 1H, aromatic H), 7.752 (d, J=8.7H₂, 2H, aromatic H), 7.869 (d, J=6.9H₂, 2H, aromatic H), 8.249(d, J=8.7H₂, 2H₂, aromatic H). FAB-MS (m/e); 329[M+H]⁺, C₁₇H₁₆N₂O₅. Calcd; C, 62.18; H, 4.91; N, 8.54. Found C, 62.30; H, 5.01; N, 8.48; mol mass: 328.32.

23: Mp,120-122° C.; IR(KBr), ν/cm⁻¹=3275(NH),3020(aromatic C═CH), 2905 and 2858 (CH and CH₂), 1625 (C═O), 1600, 1590, 1548 and 1435(aromatic C═C), 1649 and 1382 (NP2), 1188 and 1075 (C—O—C),803(1,4-disubstituted phenyl),719 and 690 (monosubstituted). ¹HNMR(CDCl₃): δ/ppm=40.301(m, 5H, NHCHCH₂O), 5.712(S, 1H, OCHO), 7,085 (d, J=6.5H₂, 1H, NH), 7.694(d, J=9.0H₂, 2H, aromatic H), 7.818(d, J=6.9H₂, 2H, aromatic H), 8.258 (d, J=9.3H₂, 2H, aromatic H), FAB-MS (m/e): 329[M+H]⁺; C₁₇H₁₆N₂O₅. Calcd, C, 62.18; H, 4.91; N, 8.54. Found, C, 62.24; H, 5.04; N, 8.48; mol mass:328.32.

### EXAMPLE 2

### The Preparation of (2S,4S,5R) and (2R,4S,5R)-2-(p-nitrophenyl)-5-benzoylamino-4-methyl-1,3-dioxane

The suspension of 100 mg (0.48 mmol) of (2S,3R)-2-benzoylamino-1,3-butylene glycol (IVb), 73 mg (0.48 mmol) of p-nitrobenzaldehyde, 10 mg of p-toluene-sulfonic acid and 100 mg of anhydrous sodium sulfate in 10 ml dichloromethane/tetrahydrofuran (5:1) was stirred at 50° C. for 12 hours, and the reaction mixture was cooled to room temperature when the TLC (chloroform/methanol, 25:1) indicated the disappearance of the raw material. The reaction mixture was neutralized using anhydrous sodium carbonate and then filtered. The filtrate was evaporated under vacuum and the resulted syrupy was separated by column chromatography (chloroform/methanol, 25:1) to give 147 mg (90%) of (2S,4Sm,5R)-2-p-nitrophenyl-5-benzoylamino-4-methyl-1,3-dioxane (25), as a colorless solid, and 8 mg (5%) of (2S,4S,5R)-2-p-nitrophenyl-5-benzoylamino-4-methyl-1,3-dioxane (24), as a colorless solid.

25: Mp,90-92° C.; IR(KBr), ν/cm⁻¹=3450(NH),3045(aromatic C═CH), 2975, 2940 and 2870(CH,CH₂ and CH₃),1625(C═O), 1600, 1576, 1480 and 1400 (aromatic C═C), 1590 and 1370 (NO₂), 1176 and 1070 (C—O—C), 795(1,4-disubstituted phenyl),694 and 741 (monosubstitutied phenyl). ¹HNMR(CDCl₃): δ/ppm=1.322 (d, J=6.2 Hz, 3H, CH₃CHO), 4.214(dt, J=3.9 Hz, 1H, NHCHCH₂O), 4.239(d, J=3.9 Hz, 2H, NHCHCH₂O), 4.296(d, J=4.8 Hz, 1H, CH₃CHO), 5.707(S, 1H, OCHO), 6.773 (d, J=9.6 Hz, 1H, NH), 7.409(t, J=1.5 Hz,2H, aromatic H), 7.435(t, J=1.8 Hz, 1H, aromatic H), 7.486(t, J=1.8 Hz,1H, aromatic H), 7.685(t, J=8.7 Hz, 1H, aromatic H), 7.848(d, J=1.5 Hz, 2H, aromatic H),8.234(d, J=5.1 Hz, 2H, aromatic H), in NOESY experiment the NOE relationships between the CH₃ at 4-position and proton of phenyl at a position, and between the CH₃ at 4-position and the NH at 5-position are observed. FAB-MS(m/e):343[M+H]⁺, [α]_{D}²⁰=−15.4 (C=0.02 in CHCl₃),C₁₈H₁₈N₂O₅. Calcd, C, 63.14; H, 5.30; N, 8.19. Found, C, 63.54; H, 5.41; N, 8.24; mol mass: 342.35.

24: mp, 112-114° C.; IR(KBr), ν/cm⁻¹=3446(NH), 3034(aromatic C═CH), 2980, 2950 and 2860 (CH,CH₂ and CH₃),1655 (C═O), 1607, 1580, 1482 and 1406 (aromatic C═C), 1595 and 1376 (NO₂), 1180 and 1068(C—O—C), 790 (1,4-disubstituted phenyl), 690 and 745 (monosubstitutied phenyl). ¹HNMR(CDCl₃): δ/ppm=1.268 (d, J=6.3 Hz, 3H, CH₃CHO), 3.930 (d, J=2.7 Hz,2H, NHCHCH₂O), 3.934(m, J=6.3 Hz, 1H, NHCHCH₂O), 4.060(m, J=3.6 Hz, 1H, CH₃CHO), 6.151(S, 1H, OCHO), 6.969 (d, J=9.6 Hz, 1H, NH), 7.481(t, J=6.6 Hz,2H, aromatic H), 7.525(t, J=7.2 Hz, 1H, aromatic H), 7.662 (d, J=8.9 Hz,2H, aromatic H), 7.856(d, J=6.3 Hz, 2H, aromatic H), 8.295(d, J=8.9 Hz, 2H, aromatic H). FAB-MS (m/e): 343[M+H]⁺, [α]_{D}²⁰=280.5 (C=0.02 in CHCl₃), C₁₈H₁₈N₂O₅. Calcd: C, 63.14; H, 5.30; N, 8.19. Found, C, 63.48; H, 5.45; N, 8.31; mol mass: 342.35.

### EXAMPLE 3

### The Preparation of (2S,5S)- and (2R,5S)-2-p-nitrophenyl-5-benzoyl-amino-1,3-dioxepane

The suspension of 150 mg (0.72 mmol) of (2s)-2-benzoylamino-1,4-butylene glycol (IVc), 108 mg (0.72 mmol) of p-nitrobenzaldehyde, 10 mg of p-nitro-benzenesulfonic acid and 100 mg of anhydrous sodium sulfate in 10 ml of dichloromethane/tetrahydrofuran (5:1) was stirred at 50° C. for 18 hours, and the reaction mixture was cooled to room temperature when the TLC (thyl acetate/petroleum ether, 5:1) indicated the disappearance of the raw material. The mixture was neutralized using anhydrous sodium carbonate and then filtered. The filtrate was evaporated under vacuum and the resulted syrupy was separated using column chromatography (chloroform/methanol, 25:1) to give 184 mg (75%) of (2S,5S)-2-p-nitrophenyl-5-benzoylaminobenzoylamino-1,3-dioxapane (26) and 25 mg (10%) of (2R,5S)-2-p-nitrophenyl-5-benzoylamino-1,3-dioxepane (27).

26:Mp, 121-123° C.; IR(KBr), ν/cm⁻¹=3301(NH),3040(aromatic C═CH), 2970, 2920 and 2850 (CH,CH₂ and CH₃), 1645 (C═O), 1609, 1562, and 1460 (aromatic C═C), 1595 and 1372 (NO₂), 1170 and 1068 (C—O—C),793 (1,4-disubstituted phenyl), 740 and 690 (monosubstitutied phenyl). ¹HNMR(CDCl₃): δ/ppm=2.140(m,2H, NHCHCH₂CH₂O), 3.760 (d, J3.0 Hz, 2H, NHCHCH₂O), 4.064 (t, J=12.1 Hz, 2H, NHCHCH₂CH₂O), 4.512 (m, 1H, NHCHCH₂O) 5.789 (S, 1H, OCHO), 6.646 (d, J=8.7 Hz, 1H, NH),7.429(t, J=7.5 Hz, 2H, aromatic H), 7.484 (t, J=7.2 Hz, 1H, aromatic H), 7.692(m, J=9.0 Hz, 4H, aromatic H), 8.223(d, J=9.0 Hz, 2H, aromatic H), in NOESY test the NOE relationships between the NH at 5 position and the protons of phenyl at 2 position are observed. FAB-MS(m/e): 343[M+H]⁺, [α]_{D}²⁰=21.0 (C=0.02 in CHCl₃), C₁₈H₁₈N₂O₅. Calcd: C, 63.14; H, 5.30; N, 8.19. Found, C, 63.25; H, 5.42; N, 8.23; mol mass: 342.35.

27:Mp, 121-123° C.; IR(KBr), ν/cm⁻¹=3310 (NH), 3051 (aromatic C═CH), 2960, 2910 and 2845 (CH, CH₂ and CH₃), 1640 (C═O), 1602, 1565, 1482 and 1456 (aromatic C═C), 1590 and 1370 (NO₂), 1165 and 1060 (C—O—C), 795 (1,4-disubstituted phenyl), 738 and 695 (monosubstituted phenyl). ¹HNMR(CDCl₃): δ/ppm=2.205 (m, 2H, NHCHCH₂CH₂O, 3.697(t, J=3.1 Hz, 2H, NHCHCH₂O), 3.897(t, J=12.0 Hz,2H, NHCHCH₂CH₂O), 3.934 (m, 1H, NHCHCH₂O), 4.054(s, 1H, OCHO), 6.896(d, J=7.8 Hz,1H, NH), 7.457 (t, J=7.5-Hz, 2H, aromatic H), 7.525(t, J=6.9 Hz,1H, aromatic H), 7.673(d, J=8.7 Hz, 2H, aromatic H), 7.815 (d, J=7.8 Hz,2H, aromatic H), 8.228(d, J=5.1 Hz, 2H, aromatic H). FAB-MS(m/e):343[M+H]⁺, [α]_{D}²⁰=−19.3(C=0.02 in CHCl₃), C₁₈H₁₈N₂O₅. Calcd: C, 63.14; H, 5.30; N, 8.19. Found, C, 63.28; H, 5.37; N, 8.26; mol mass:342.35.

### EXAMPLE 4

### The Preparation of (2S,5S)- and (2R,5S)-2-(p-nitrophenyl)-5-benzoyl-1,3-dioxacyclooctane

The suspension of 106 mg (0.47 mmol) of (2S)-2-benzoylamino-1,5-pentanediol (IVd), 72 mg (0.47 mmol) of p-nitrobenzaldehyde, 10 mg of p-nitrobenzene-sulfonic acid, and 100 mg of anhydrous sodium sulfate in 20 ml dichloromethane/tetrahydrofuran (5:1) was stirred at 50° C. for 12 hours, and the reaction mixture was cooled to room temperature when the TLC (chloroform/methanol, 25:1) indicated the disappearance of the raw material. The reaction mixture was neutralized using anhydrous sodium carbonate and then filtered. The filtrate was evaporated under vacuum and the resulted residue was separated using column chromatography (chloroform/methanol, 25:1) to give 108 mg (65%) of (2S,5R)-2-p-nitrophenyl-5-formamide-1,3-dioxacyclooctane (28) as a colorless solid and 13 mg (8%) of (2R,5S)-2-p-nitrophenyl-5-benzoylamino-1,3-dioxacyclooctane (29) as a colorless solid.

28:Mp, 132-133° C.; IR(KBr), ν/cm⁻¹=3310(NH), 3050(aromatic C═CH), 2930, and 2850(CH and CH₂), 1630 (C═O),1605, 1580, 1505 and 1450 (aromatic C═C), 1650 and 1386 (NO₂), 1190 and 1070 (C—O—C),801 (1,4-disubstituted phenyl), 718 and 680 (monosubstituted phenyl). ¹HNMR(CDCl₃): δ/ppm=1.821(m, 4H, NHCHCH₂CH₂CH₂O), 3.863(d,J=6.9 Hz, 1H,NHCHCH2O), 3.955(d, J=6.9 Hz,1H, NHCHCH₂O), 3.963 (m, J=5.4 Hz,1H, NHCHCH₂O), 4.303(m, J=6.9 Hz, 2H, NHCHCH₂CH₂CH₂O), 5.834(s, 1H, OCHO), 6.670(d, J=6.9 Hz, 1H, NH), 7.408(t, J=7.8 Hz,2H, aromatic H), 7.437(t, J=7.2 Hz, 1H, aromatic H), 7.601 (d, J=9.0 Hz,2H, aromatic H), 7.631(d, J=9.0 Hz, 2H, aromatic H), 8.204(d, J=8.4 Hz, 2H, aromatic H). FAB-MS(m/e):357[M+H]⁺, [α]_{D}²⁰=20.0(C=0.02 in CHCl₃), C₁₉H₂₀N₂O₅. Calcd: C, 64.02; H, 5.66; N, 7.86. Found, C, 64.22; H, 5.70; N, 7.92; mol mass:356.38.

29:Mp, 116-118° C.; IR(KBr), ν/cm⁻¹=3318(NH), 3060(aromatic C═CH), 2950, and 2860 (CH and CH₂), 1620 (C═O),1610, 1590, 1500 and 1460 (aromatic H), 1660 and 1383 (NO₂), 1119 and 1090 (C—O—C), 800 (1,4-disubstituted phenyl), 718 and 690 (monosubstituted phenyl). ¹HNMR(CDCl₃): δ/ppm=1.852(m, 4H, NHCHCH₂CH₂CH₂O), 3.771 (d, J=6.9 Hz, 2H, NHCHCH₂O), 3.902(m, J=6.3 Hz,1H, NHCHCH₂O), 4.066 (m, P=6.7 Hz,2H, NHCHCH₂CH₂CH₂O), 5.720(s, 1H, OCHO), 6.383 (d, J=6.0 Hz, 1H, NH), 7.240 (t, J=6.9 Hz,2H, aromatic H), 7.517(t, J=7.5 Hz, 1H, aromatic H), 7.706 (d, J=7.2 Hz,2H, aromatic H), 8.022(d, J=6.6 Hz, 2H, aromatic H), 8.193(d, J=8.7 Hz, 2H, aromatic H). FAB-MS(m/e):357[M+H]⁺,[α]_{D}²⁰=−180.0(C=0.02 in CHCl₃), C₁₉H₂₀N₂O₅. Calcd: C, 64.02; H, 5.66; N, 7.86. Found: C, 64.28; H, 5.79; N, 7.98; mol mass:356.38

### EXAMPLE 5

### The Preparation of (cis)-2-p-aminophenyl-5-benzoylamino-1,3-dioxane

To the solution of 78.8 mg (0.24 mmol) of (cis)-2-(p-nitrophenyl)-5-benzoylamino-1,3-dioxane in 10 ml of anhydrous alcohol 10 mg of pd/C (5%) was added and the reaction mixture was aerated with hydrogen gas for 12 hours, and the reaction mixture was cooled to room temperature when the TLC (chloroform/methanol, 15:1) indicated the disappearance of the raw material. The reaction mixture was filtered and the filtrate was evaporated under vacuum and the residue was purified using column chromatography (chloroform/methanol, 15:1) to give 61 mg (85%) of (cis)-2-(p-aminophenyl)-5-benzoylamino-1,3-dioxane (30) as a brown syrupy.

30: IR(smear), ν/cm⁻¹=3320, 3370 and 3270(NH), 3020(aromatic C═CH), 2930 and 2860(CH and CH₂), 1625 (C═O), 1605, 1585, 1540 and 1460 (aromatic C═C), 1190 and 1065 (C—O—C), 825 (1,4-disubstituted phenyl), 710 and 685 (monosubstituted phenyl). ¹HNMR(DMSO): δ/ppm=4.226 (d, J=7.5 Hz, 2H, NHCHCH₂O), 4.306(d, J=7.5 Hz, 2H, NHCHCH₂O),4.325(m, 1H, NHCHCH₂O), 4.785(s,2H,NH₂), 5.705(s, 1H, OCHO), 7.350(t, J=6.4 Hz, 2H, aromatic H), 7.537(t, J=6.4 Hz, 1H, aromatic H), 7.720 (d, J=8.5 Hz,2H, aromatic H), 7.843(d, J=6.7 Hz, 2H, aromatic H), 8.230(d, J=8.5 Hz, 2H, aromatic H). FAB-MS (m/e): 209[M+H]⁺, C₁₇H₁₈N₂O₃. Calcd: C, 68.44; H, 6.08; N, 9.39. Found: C, 68.60; H, 6.22; N, 9.10.

### EXAMPLE 6

### The Preparation of (2S,4S,5R)-2-p-aminophenyl-5 benzoylamino-4-methyl-1,3-dioxane

To the solution of 115 mg (0.34 mmol) of (2S,4S,5R)-2-p-nitrophenyl-5-benzoylamino-4-methyl-1,3-dioxane in 10 ml of anhydrous alcohol 10 mg of pd/C (5%) was added. The reaction mixture was aerated with hydrogen gas for 12 hours and then cooled to room temperature when the TLC (chloroform/methanol, 15:1) indicated the disappearance of the raw material. The reaction mixture was filtered and the filtrate was evaporated under vacuum and the resulted residue was purified using column chromatography (chloroform/methanol, 15:1) to give 91 mg (90%) of (2S,4S,5R)-2-p-aminophenyl-5-benzoyl-amino-4-methyl-1,3-dioxane (31) as a brown syrupy.

31: IR(smear), ν/cm⁻¹=3425, 3369 and 3269(NH), 3034(aromatic C═CH), 2980, 2940 and 2875(CH, CH₂ and CH₃), 1660 (C═O),1605, 1578, 1480 and 1410 (aromatic C═C), 1189 and 1060 (C—O—C), 829 (1,4-disubstituted phenyl), 708 and 680 (monosubstituted phenyl). ¹HNMR (CDCl₃): δ/ppm=1.270(d, J=6.1 Hz, 3H, CH₃CHO),3.952 (d, J=2.9 Hz; 2H, NHCHCH₂O), 3.960(m, J=6.2 Hz, 1H, NHCHCH₂O), 4.069(m, J=3.8 Hz, 1H, CH₃CHO),5.001(s, 2H, NH₂), 6.150(s, 1H, OCHO), 6.945(d, J=9.4 Hz, 1H, NH), 7.462(t, J=6.4 Hz, 2H, aromatic H), 7.520(t, J=7.0 Hz, 1H, aromatic H), 7.660 (d, J=8.7 Hz, 2H, aromatic H), 7.852(d, J=6.4 Hz, 2H, aromatic H), 8.292(d, J=8.7 Hz, 2H, aromatic H). FAB-MS (m/e): 313[M+H]⁺, [α]_{D}²⁰=29.4(C=0.02 in CHCl₃), C₁₈H₂₀N₂O₃. Calcd: C, 69.21; H, 6.45; N8.97. Found: C, 69.50; H, 6.65; N, 8.72.

### EXAMPLE 7

### The Preparation of (2S,5S)-2-(p-aminophenyl)-5-benzoylamino-1,3-dioxapane

To the solution of 116 mg (0.34 mmol) of (2S,5S)-2-(p-nitrophenyl)-5-benzoylamino-1,3-dioxapane in 10 ml of anhydrous alcohol 10 mg of pd/C (5%) was added. The reaction mixture was aerated with hydrogen gas for 12 hours and then cooled to room temperature when the TLC (chloroform/methanol, 15:1) indicated the disappearance of the raw material. The reaction mixture was filtered and the filtrate was evaporated under vacuum and the resulted residue was purified using column chromatography (chloroform/methanol, 15:1) to give 92 mg (87%) of (2S,5S)-2-p-aminophenyl-5-benzoylamino-1,3-dioxapane (32) as a brown syrupy.

32:IR(KBr smear): ν/cm⁻¹=3430, 3365 and 3260 (NH), 3040 (aromatic C═CH),2925 and 2849(CH and CH₂), 1638 (C═O), 1608, 1575, 1509 and 1460 (aromatic C═C), 1180 and 1065 (C—O—C), 830 (1,4-disubstituted phenyl), 710 and 675 (monosubstituted phenyl). ¹HNMR(CDCl₃): δ/ppm=1.830(m, 4H, NHCHCH₂CH₂CH₂O), 3.901 (d, J=6.8 Hz, 1H, NHCHCH₂O), 3.959 (m, J=6.8 Hz, 1H, NHCHCH₂O), 3.965(m, J=5.4 Hz, 1H, NHCHCH₂O), 4.310(m, J=6.8 Hz, 2H, NHCHCH₂CH₂CH₂O), 5.004 (s, 2H, NH₂), 5.830 (s, 1H, OCHO), 6.675 (d, J=6.8 Hz, 1H, NH), 7.490 (t, J=7.6 Hz, 2H, aromatic H), 7.434 (t, J=7.0 Hz, 1H, aromatic H), 7.596 (d, J=9.0 Hz, 2H, aromatic H), 7.628 (d, J=9.0 Hz, 2H, aromatic H), 8.200(d, J=8.4 Hz, 2H, aromatic H). FAB-MS(m/e):327[M+H]⁺,[α]_{D}²⁰=20.9(C=0.02 in CHCl₃), C₁₉H₂₂N₂O₃. Calcd: C, 69.92; H, 6.79; N, 8.58. Found: C, 69.69; H, 6.48; N, 8.71.

### EXAMPLE 8

### The Preparation of (cis) and (trans)-2-(E) phenylpropenyl-5-benzoylamino-1,3-dioxane:

The suspension of 135 mg (0.69 mmol) of 2-benzoylamino-1,3-propylene glycol (IVa), 92 mg (0.69 mmol) of phenyl acrylaldehyde, 10 mg of p-toluenesulfonic acid and 100 mg of anhydrous sodium sulfate in 20 ml of chloroform was stirred at 50° C. for 12 hours and then cooled to room temperature when the TLC (ethyl acetate/petroleum ether,1:2) indicated the disappearance of the raw material. The reaction mixture was neutralized using anhydrous Na₂CO₃ and then filtered. The filtrate was evaporated under vacuum and the resulted residue was separated using column chromatography (ethyl acetate/petroleum ether, 1:2) to give 158 mg (71%) of (cis)-2-(E)-phenylpropenyl-5-benzoylamino-1,3-dioxane (34) as a colorless solid and 49 mg (22%) of (trans)-2-(E)-phenylpropenyl-5-benzoylamino-1,3-dioxane (35) as colorless solids.

34:Mp, 119-122° C., IR(KBr): ν/cm⁻¹=3450 (NH), 3025 (aromatic and olefinic C═CH),2950 and 2830 (CH and CH₂), 1630 (C═O), 1600, 1580, 1500 and 1460 (aromatic and olefinic C═C), 1190 and 1070 (C—O—C), 740 and 690 (monosubstituted phenyl). ¹HNMR(CDCl₃): δ/ppm=4.032(m, J=6.5 Hz, 1H, NHCHCH₂O), 4.175 (d, J=6.4 Hz, 4H, NHCHCH₂O), 5.526 (d, J=3.3 Hz, 1H, OCHO), 6.204(d, J=16.2 Hz, 1H, CH═CH—C₆H₅),6.814(d, J=16.2 Hz, 1H, CH═CH—C₆H₅), 7.185 (d, J=4.5 Hz, 1H, NH), 7.287(t, J=6.0 Hz, 1H, aromatic H), 7.336 (t, J=7.5 Hz, 2H, aromatic H), 7.409 (d, J=6.6 Hz, 2H, aromatic H), 7.466 (t, J=5.4 Hz, 2H, aromatic H), 7.844(d, J=6.6 Hz, 2H, aromatic H). FAB-MS(m/e): 324[M+H]⁺, C₂₀H₂₁NO₃. Calcd: C, 74.27; H, 6.55; N, 4.33. Found: C, 74.35; H, 6.61; N, 4.38. mol mass:323.39.

35:Mp, 115-118° C., IR(KBr): ν/cm⁻¹=3460 (NH), 3030 (aromatic and olefinic C═CH), 2960 and 2830 (CH and CH₂), 1632 (C═O), 1605, 1590, 1501 and 1485(aromatic and olefinic C═C), 1185 and 1069 (C—O—C), 745 and 691 (monosubstituted phenyl). ¹HNMR(CDCl₃): δ/ppm=3.687(m, J=6.5 Hz, 1H, NHCHCH₂O),4.134 (d, J=6.9 Hz, 4H, NHCHCH₂O), 5.123 (d, J=4.3 Hz, 1H, OCHO), 6.134(d, J=16.2 Hz, 1H, CH═CH—C₆H₅),6.823 (d, J=16.2 Hz, 1H, CH═CH—C₆H₅), 7.299 (t, J=6.0 Hz, 1H, aromatic H), 7.338(t, J=6.0 Hz, 2H, aromatic H), 7.434 (d, J=6.9 Hz, 1H, aromatic H), 7.451 (t, J=8.7 Hz, 2H, aromatic H), 7.510 (t, J=6.9 Hz, 1H, aromatic H), 7.772(d, J=7.5 Hz, 2H, aromatic H). FAB-MS(m/e): 324[M+H]⁺, C₂₀H₂₁NO₃. Calcd: C, 74.27; H, 6.55; N, 4.33. Found: C, 74.32; H, 6.55; N, 4.41. mol mass:323.39.

### EXAMPLE 9

### The Preparation of (2S,4S,5R)- and (2R,4S,5R)-2-(E)-phenylpropenyl-5-benzoylamino-1,3-dioxane:

The suspension of 128 mg (0.61 mmol) of 2-benzoylamino-1,3-butylene glycol (IVb), 82 mg (0.61 mmol) of phenyl acrylaldehyde, 10 mg of p-toluenesulfonic acid, and 100 mg of anhydrous sodium sulfate in 20 ml chloroform was stirred at 50° C. for 16 hours and then cooled to room temperature when the TLC (ethyl acetate/petroleum ether,1:1) indicated the disappearance of the raw material. The resulted residue was separated using column chromatography (ethyl acetate/petroleum ether,1:2) to give 179 mg (91%) of (2S,4S,5R)-2-(E)-phenylpropenyl-5-benzoylamino-4-methyl-1,3-dioxane (36) and 17 mg (9%) of (2R,4S,5R)-2-(E)-phenylpropenyl-5-benzoylamino-4-methyl-1,3-dioxane (37) as colorless solids.

36:Mp, 123-125° C., IR(KBr): ν/cm⁻¹=3269 (NH), 3010(aromatic and olefinic C═CH), 2936, 2860 and 2830 (CH CH₂ and CH₃), 1625 (C═O), 1605, 1590, 1550 and 1480 (aromatic and olefinic C═C), 1386(CH₃), 1189 and 1080 (C—O—C), 720 and 685 (monosubstituted phenyl). ¹HNMR(CDCl₃): δ/ppm=1.218(d, 3 J=6.0 Hz, 3H, CH₃),3.654(m, J=6.5 Hz, 1H, NHCHCH₃), 3.875(m, J=6.6 Hz, 1H, CH₃CHO), 4.335(d, J=6.9 Hz, 2H, NHCHCH₂O), 4.346 (d, J=5.7 Hz, 1H, OCHO), 6.366 (d, J=15.9 Hz, 1H, CH═CH—C₆H₅), 6.575 (d, J=15.9 Hz, 1H, CH═CH—C₆H₅), 7.221 (d, J=6.8 Hz, 1H, NH), 7.244(t, J=6.9 Hz, 4H, aromatic H), 7.364 (d, J=6.9 Hz, 2H, aromatic H), 7.405 (d, J=6.9 Hz, 2H, aromatic H). FAB-MS(m/e): 324[M+H]⁺, C₂₀H₂₁NO₃. Calcd: C, 74.27; H, 6.55; N, 4.33. Found: C, 74.35; H, 6.61; N, 4.38. mol mass:323.39.

37:Mp, 115-118° C., IR(KBr): ν/cm⁻¹=3245(NH), 3024(aromatic and olefinic C═CH), 2930, 2860 and 2810 (CH CH₂ and CH₃), 1620(C═O), 1601, 1585, 1560 and 1460 (aromatic and olefinic C═C), 1380(CH₃), 1190 and 1076(C—O—C), 724 and 680 (monosubstituted phenyl). ¹HNMR(CDCl₃): δ/ppm=1.220(d, J=6.0 Hz, 3H, CH₃), 3.641(m, J=6.3 Hz, 1H, NHCHCH₂O),4.119(m, J=9.6 Hz, 2H, NHCHCH₂O), 4.305(m, J=6.5 Hz, 1H, NHCHCH₂O), 5.298(d, J=5.2 Hz, 1H, OCHO), 6.242 (d, J=16.4 Hz, 1H, CH═CH—C₆H₅),6.840(d, J=16.4 Hz, 1H, CH═CH—C₆H₅), 7.219 (d, J=6.5 Hz, 1H, NH), 7.295(t, J=6.6 Hz, 4H, aromatic H), 7.412 (t, J=6.2 Hz, 2H, aromatic H), 7.507 (d, J=6.6 Hz, 2H, aromatic H),7.833 (d, 6.3, 2H, aromatic H). FAB-MS(m/e): 324[M+H]⁺, C₂₀H₂₁NO₃. Calcd: C, 74.27; H, 6.55; N, 4.33. Found: C, 74.34; H, 6.64; N, 4.36. mol mass:323.39.

### EXAMPLE 10

### The Evaluation of Anti-Inflammatory Activity

An adaptation of the method of Gad et al (1986) was employed for sensitization. Mice were allowed to acclimate for a week after arrival in the laboratory, then were screened to remove any from testing which have ears that appear red or swollen. Male Kunming species, 6 to 8 weeks old, about 20-25 g, were shaved and tape stripped at the start of each study. Mice were randomly divided into test group, vehicle control group, and positive control group with 10 mice each one.

The mice in vehicle control group were administered orally 0.2 ml of carboxymethyl cellulose (0.5%), while the mice in the positive control group were administered orally 0.2 ml of suspension of aspirin in carboxymethyl cellulose (0.5%) at a dosage of 30 mg/kg, and a concentration of 0.3 mg/ml, while the mice in the test group were administered orally 0.2 ml of suspension of the test compound of the present invention in carboxymethyl cellulose (0.5%) at a dosage of 30 mg/kg, and a concentration of 0.3 mg/ml. 30 minutes later, 0.02 ml of xylene in solution was applied to the center of the shaved region on the left ear of each animal (test and control) and the same was applied to the right ear. The application was allowed to dry before the animal was returned to its cage. Mice were killed 4 hours later, and a 9-mm Keyes cutaneous punch was used to obtain uniform tissue sections for weight variation which indicating the swelling degree. Groups were statistically analyzed using the Wilk-Shapiro test for normality and the paired parametric Student t test with a significance level of p<0.05. Data were expressed as the mean difference plus or minus the standard error. The results are shown below:

1. Multiple Administration

Each mouse in the vehicle control mice group was administered orally 0.2 ml of carboxymethyl cellulose (0.5%) once a day, while each mouse in the positive control group was administered orally suspension of 0.6 mg of aspirin in 0.2 ml of carboxymethyl cellulose (0.5%) once a day, and each mouse in the test group was administed orally the suspension of 0.6 mg of the compounds of the present invention in 0.2 ml of carboxymethyl cellulose (0.5%) once a day. The applications were repeated for three consecutive days. The weight variation is listed in Table 1.

TABLE 1
The weight variation after 3 consecutive days test

| | Weight variation |
|---|---|
| Comp. | (X ± SD) mg |
| control | 12.01 ± 3.96 |
| Aspirin | 4.26 ± 1.44¹⁾ |
| 1 | 8.63 ± 3.13 |
| 3 | 5.74 ± 1.71²⁾ |
| 5 | 5.89 ± 2.03^{2,3)} |
| 8 | 4.08 ± 1.77^{1,4)} |
| 9 | 3.97 ± 1.49¹⁾ |
| 16 | 3.84 ± 1.99¹⁾ |

Dosage = 30 mg/Kg, n = 10;
¹⁾compared with vehicle control group, P < 0.001;
²⁾compared with vehicle control group, P < 0.01;
³⁾compared with 1, P < 0.05;
⁴⁾compared with 1, P < 0.001.

2. Administered for 1 Time

Administered with the same manner and dosage. The weight variation was determined, and the result is shown in Table 2.

TABLE 2
The weight variation after administered for 1 time

| | Dosage | Weight variation |
|---|---|---|
| Comp. | (mg/kg) | (X ± SD mg) |
| Control | | 7.76 ± 1.55 |
| Aspirin | 30 | 4.17 ± 1.80¹⁾ |
| 1 | 10 | 2.62 ± 1.37^{1,6)} |
| 2 | 10 | 3.58 ± 1.70^{2,4)} |
| 3 | 10 | 3.73 ± 1.11³⁾ |
| 5 | 30 | 3.39 ± 2.28^{3,4)} |
| 9 | 30 | 3.28 ± 2.06^{3,5)} |
| 15 | 10 | 2.22 ± 1.41^{3,6)} |
| 16 | 30 | 4.23 ± 2.46¹⁾ |
| 17 | 30 | 7.97 ± 4.36 |
| 18 | 21 | 4.31 ± 1.75¹⁾ |
| 38 | 30 | 5.81 ± 2.46 |
| 39 | 30 | 4.84 ± 2.30¹⁾ |
| 40 | 21 | 3.55 ± 1.69^{3,6)} |

n = 10
¹⁾compared with vehicle control group, P < 0.05;
²⁾compared with vehicle control group, P < 0.01;
³⁾compared with vehicle control group, P < 0.001;
⁴⁾compared with Aspirin, P < 0.05;
⁵⁾compared with Aspirin, P < 0.01
⁶⁾compared with Aspirin, P < 0.001.

3. Dosage-Effect Relationship

The dosage-effect relationship of the excellent anti-inflammatory compounds were determined, and the result is listed in Table 3.

TABLE 3
Dosage-effect relationship of some compounds

| | Dosage | Weight variation |
|---|---|---|
| Comp. | (mg/Kg) | (X ± SD)mg |
| Control | | 6.29 ± 2.07 |
| 1 | 10 | 1.90 ± 0.99 |
| | 5 | 2.64 ± 1.49 |
| | 3.5 | 3.16 ± 1.07 |
| 5 | 30 | 2.76 ± 1.85 |
| | 21 | 3.87 ± 1.06 |
| | 3.5 | 5.01 ± 1.81 |
| 16 | 30 | 3.44 ± 2.00 |
| | 10 | 4.10 ± 3.07 |
| | 3.5 | 5.82 ± 1.47 |
| 24 | 30 | 1.43 ± 0.89 |
| | 7 | 1.81 ± 0.63 |
| | 2.3 | 3.21 ± 1.68 |
| 42 | 30 | 0.69 ± 0.33 |
| | 21 | 0.86 ± 0.40 |
| 34 | 30 | 1.54 ± 1.09 |
| | 21 | 2.17 ± 0.68 |
| | 7 | 2.61 ± 1.49 |

1. The application of a compound selected from the group consisting of 5-benzoylamino-1,3-dioxacyclane derivatives represented by the following formulae 1-48 in preparing protein kinase inhibitors:

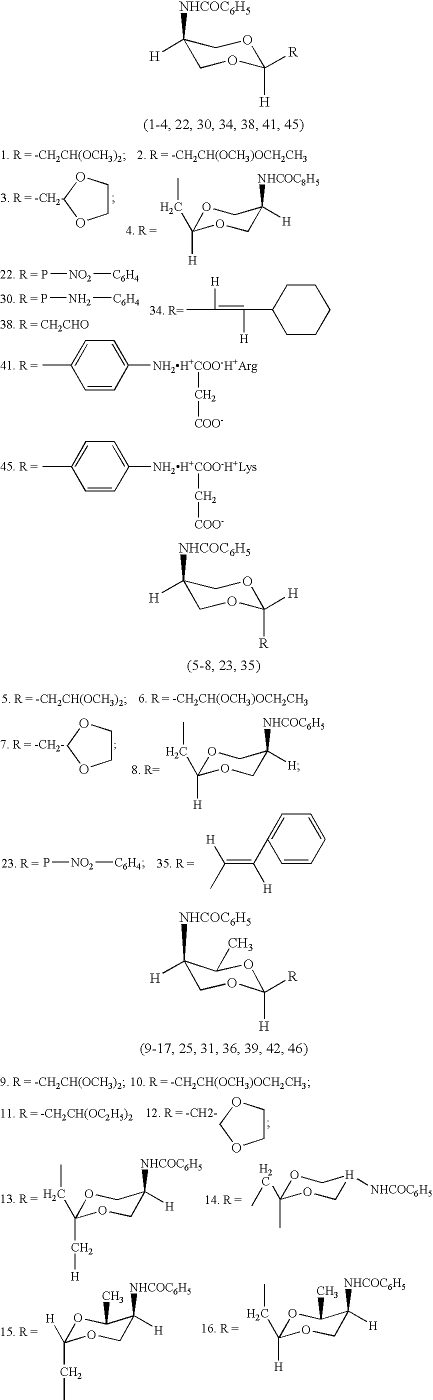

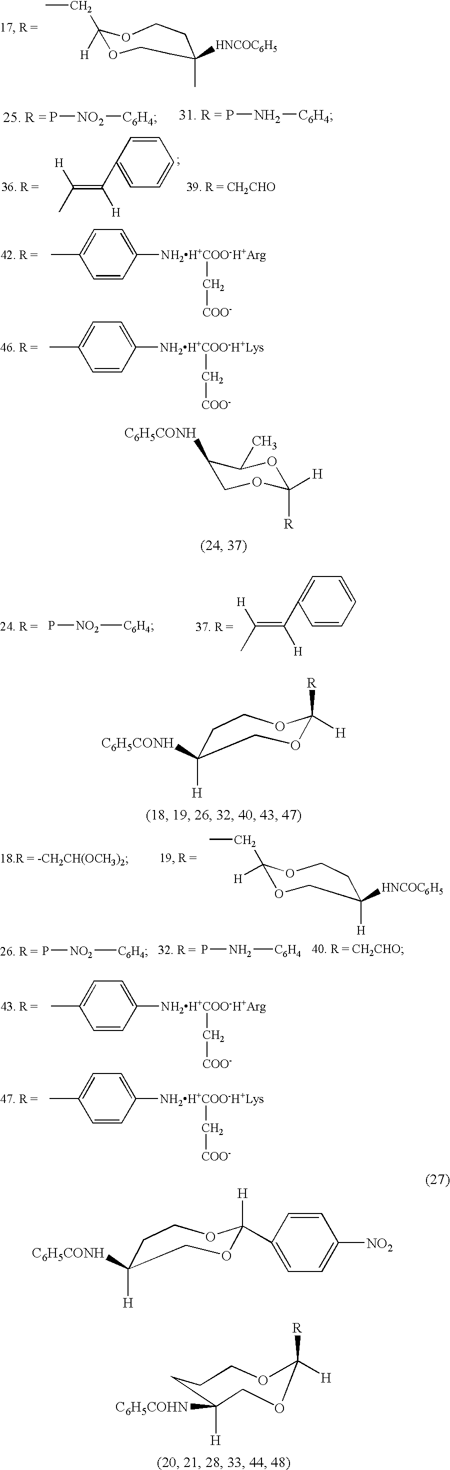

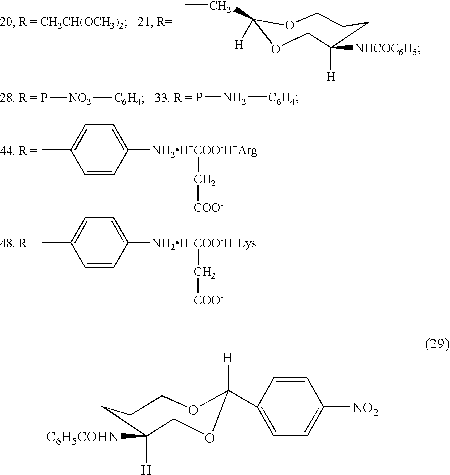

2. A compound selected from the group consisting of 5-benzoylamino-1,3-dioxacyclane derivatives represented by the formulae 22-48 described in claim 1.

3. A method of preparing the compound described in claim 2, which including a stereo-specific acetal transfer reaction between N-benzoylamino glycol and aromatic aldehyde.

4. The method of claim 3, wherein said stereo-specific acetal transfer reaction includes: preparing benzoylamino glycol (include optically active glycols) via using L-amino acid as raw material of a methyl esterification, benzoylation, and reducing reaction, and cyclizing with p-nitrobenzaldehyde or phenylacrylaldehyde to produce cylized product.

5. The method of claim 4, wherein the nitro groups in said cylized product is reduced to amino and the reduced product is obtained.

6. The method of claim 5, wherein said reduced product react with propane diacid and basic amino acid in sequence to be salified.

7. The method of claim 6, wherein said basic amino acid is L-Arg or L-Lys.
